(19) United States
(12) Patent Application Publication
(10) Pub. No.: US 2019/0008732 A1
(43) Pub. Date: Jan. 10, 2019

(54) COSMETIC COMPOSITIONS FOR PROTECTION OF FEET FROM HOT SURFACES

(71) Applicant: Max Tucker, Penn Valley, PA (US)

(72) Inventors: Max Tucker, Penn Valley, PA (US); Robert J. Verdicchio, Bridgewater, NJ (US)

(21) Appl. No.: 16/031,116

(22) Filed: Jul. 10, 2018

Related U.S. Application Data
(60) Provisional application No. 62/530,685, filed on Jul. 10, 2017.

(51) Int. Cl. A61K 8/04; A61Q 17/04; A61K 8/81; A61K 8/31; A61K 8/49; A61K 8/34; A61K 8/87; A61K 8/92; A45D 34/04

(52) U.S. Cl. CPC A61K 8/046; A61K 2800/244; A61Q 17/04; A61K 8/8135; A61K 8/31; A61K 8/4973; A61K 8/34; A61K 8/8129; A61K 8/87; A61K 8/922; A61K 8/927; A45D 34/041; A45D 34/042; A61K 2800/874; A61K 8/042

(57) ABSTRACT

This invention describes thermal insulative compositions and methods for protecting feet from hot surfaces particularly sand, concrete, and asphalt found on beaches. The compositions comprise a film forming matrix which may or may not be polymeric; a phase transition compound such as wax, nonionic surfactants and a solvent. The thermal insulative compositions have a first state in a liquid, cream, or gel form, and a second state in a solid film form.

### CROSS-REFERENCE TO RELATED APPLICATIONS

This application claims the benefit of U.S. Provisional Patent Application No. 62/530,685 which was filed Jul. 10, 2017, the disclosures of which are hereby incorporated by reference.

### BACKGROUND OF THE INVENTION

The present invention relates generally to the cosmetic compositions providing thermal protective barriers. More particularly, the present invention relates to a cosmetic composition configured to provide a thermal protective barrier between the bottom of the foot and a heated surface.

Beaches and stone surfaces are feet receiving surfaces which can be externally heated via sunlight to temperatures capable of burning the skin of feet. These temperatures, which can exceed 110° F., are difficult if not impossible to navigate without proper footwear. The latter can be cumbersome and uncomfortable particularly on sand or water where it is desirable to navigate and swim without footwear. Pools where swimming areas are lined with stone or cement also hold sufficient heat to affect and burn the feet making recreational swimming a less than a pleasant task.

The outer layers of the skin become thinner as a result of the aging process, owing to decreases in elastin and collagen. As such, the skin becomes more sensitive to heat and other forces. Thus, during recreational activities such as swimming, it is desirable to cover and protect the skin from the traumatic effects of heat. In spite of formal beach footwear like sandals and water shoes being available, some people are unwilling to use these beach footwear solutions due to their weight and the inconvenience of frequent putting them on and removing them. In addition, less fortunate consumers may not have sufficient funds or may lack interest in purchasing additional footwear for summer recreation.

In some cases people do not like to wear shoes/sandals on a beach because the latter are uncomfortable. Sandal type beachwear may flip around on uneven surfaces, get stuck, and often fall off during walking and/or during most any recreational movement or exercise modalities employed as part of the swimming experience. As a result attempts have been made to hold footwear more firmly to feet to provide protection, comfort, and ease of use while employed during the foregoing activity(ies).

Based on the foregoing, considerable effort has been made to optimize footwear or beachwear and other recreational wear where shoes and/or sneakers are not warranted. Sandals with removable straps connected to orifices which clamp the foot firmly to the sole are described in U.S. Pat. No. 8,819,964 B2. This still requires considerable efforts by the consumer to protect feet from heated surfaces. Similarly disposable foot protection cover/devices are described in U.S. Pat. No. 8,460,789 whereby feet are protected using paper disposable covers which employ a reusable adhesive in order to attach the cover to feet. An interesting improvement to flip flop sandals is disclosed in U.S. Pat. No. 9,603,409 which describes a method of interchangeable straps and laces. The ease of interchangeability is claimed to provide an advantage over the prior art.

In addition, there are myriad cosmetic and pharmacological emulsions, sprays, and compositions designed to treat and/or mitigate symptoms or problems sustained by feet. For example, U.S. Pat. No. 9,668,957 describes wax emulsions containing a malodor inhibitor for use on skin and feet. None of the aforementioned inventions or disclosures describe a convenient method of foot protection to guard against heated surfaces and/or stone and concrete surfaces and most importantly hot sand from beach swimming and recreation.

Thus the need exists to provide a consumer article of footwear which is easily applied and protects skin and particularly feet from hot surfaces. Described herein are both compositions and method of a foot protective article which is effective and easy for consumer use.

### SUMMARY OF THE INVENTION

The present invention provides thermal insulative compositions and methods of applying the compositions directly onto skin and feet to protect against heated surfaces such as sand, asphalt or concrete. In some embodiments the thermal insulative composition may also be water resistant or repellant such that the composition remains on the wearer's feet while swimming or walking through wet areas. Such compositions may primarily comprise a film forming agent, a phase transitioning/changing material (PTM/PCM), at least one surface active wetting agent or surfactant, and a solvent.

In some embodiments of the present invention the composition comprises a first state in liquid, gel, or cream form and a second state in solid form. The first state is useful in storing the composition before it is topically applied to the skin and dried into a second state as a solid film. The composition in a first state may be stored in a container and be dispensed and applied therefrom. In some embodiments, the container may be a bottle with a brush, or a brush coupled to a cap on the bottle. In some embodiments, the bottle may be similar to that of a roll-on deodorant container, namely having a ball used to roll on the composition from the bottle onto the user's skin. In other embodiments, the composition may be stored in a spray bottle with a conventional pumping mechanism or in a pressurized aerosol can.

A method for protecting skin from thermal damage using the thermal insulative composition in one embodiment may include rubbing, brushing, rolling or spraying the composition in the first state topically onto the skin and allowing the composition to dry into a second solid film state. For additional protection, the application steps may be repeated multiple times to form multiple film layers on the skin. The film layer may then later be removed by peeling it off or washing it off.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### DETAILED DESCRIPTION OF THE PREFERRED EMBODIMENTS

The foregoing and other features and advantages of the invention will become more apparent from the following detailed description of exemplary embodiments, read in conjunction with the accompanying drawings. The detailed description and drawings are merely illustrative of the invention rather than limiting, the scope of the invention being defined by the appended claims and equivalents thereof.

The inventive composition primarily comprises a film forming agent, a phase transitioning/changing material (PTM/PCM), at least one surface active wetting agent or surfactant, and a solvent. The composition in a first state is in liquid, gel, or cream form and a second state in solid form. The first state is useful in storing the composition before it is topically applied to the skin and dried into a second state as a solid film. Upon application, the first state coatings of the composition may dry to a homogeneous self-adhesive film. The film coating helps to protect the skin at the bottom of the user's feet by providing a layer of thermal insulation between heat source and user. In some embodiments the user may experience low amounts of heat transfer allowing the user to comfortably walk on surfaces ranging from 110° F. to 140° F. or even up to temperatures of 200° F. depending on the composition formation and the number of layers applied to the skin. The composition may have a thermal resistivity configured to keep the skin facing side under 110° F. or at ambient temperature while the environment facing side may be exposed to temperatures as discussed above.

The film forming agent of the composition acts within the composition to form a solid second state film onto the skin of a user when the composition is topically applied and allowed to dry. The film forming agent may comprise polyacetates, polyamides, polyacrylates, polyvinyl alcohol modified rosins, butadiene/styrene linear copolymers, and polyurethanes. The foregoing can be used alone or in combination as required to provide a matrix capable of embedding heat resistant compounds.

The phase transition/change materials (PTM/PCM) of the composition act as heat absorbing insulators which maintain ambient temperatures during phase transition. Said PTMs maintain constant temperature while absorbing heat until phase change is complete. There are thousands of materials and compounds classified as PTMs with many applications. Examples include, but not limited to, fatty acids hydrocarbons, inorganic salt hydrates such as glaubers salt, and waxes. The latter can be tailored to maintain any temperature range. Examples of waxes employed, but not limited to, in this invention are paraffin, carnauba, beeswax, ceresin, polyethylene waxes, and mixtures thereof. Combinations of PTMs will become evident to those skilled in the art going forward and as a result any PTM or combination(s) of the latter are within the scope of this invention. As one of skill in the art would recognize, any film forming resin or polymer which is capable of forming a matrix for said PTMs are also suitable.

Removability of the composition once applied and in solid state is accomplished using surfactants in order to promote de-adhesion of the film. Virtually any surfactant can be employed as de-adhesion is an inherent property of the latter. However the surfactant system must be tailored to allow adhesion during use while facilitating peel off/removal after use. To this effect, mild nonionic surfactants such as polysorbates are preferred embodiments for use in this invention, although any compatible nonionic surfactant can utilized. These may comprise of Spans and Tweens, which can be used alone or in combination. Said nonionic surfactants, when used in combination are combined at 1-10 to 10-1 by weight ratio in the compositions herein and preferably 1-2 to 2-1. The total weight of nonionic surfactant should not exceed 20% by weight either alone or in combination. The vehicle can be water, hydro alcoholic or alcohol alone, the latter being most preferred for drying time. Colorants fragrances and preservative(s) and/or antioxidants can be added if desired. The scope of this invention will become obvious in the light of the following examples.

### EXAMPLES

### Example 1 Composition and Process Concentrate

| INGREDIENT | WT % | WT/g |
|---|---|---|
| ETHANOL | 48.00 | 6.00 |
| GIOVEREZ P-0580 (Polyurethane-21) 33% | 40.00 | 5.00 |
| KAHL WAX 2442 P 5 (Carnauba wax) | 12.00 | 1.50 |
| TOTAL | 100.00 | 12.50 |

### Procedure

1. ADD SELVOL TO GIOVEREZ WITH MIXING UNTIL UNIFORM

2. ADD ETOH, MIX

3. ADD WAX, MIX UNTIL UNIFORM

4. ADD SURFACTANT, MIX

5. QS WITH ETOH

### Example 2

The composition of Example 1 was padded onto a glass slide and air dried for 10-15 minutes to provide an adhesive film to coat the slide. The coated slide and untreated slide were placed on a hot plate heated to 60° C. The treated slide remained cool to the touch while the untreated was too hot to touch. The experiment was repeated after 30 minutes with similar results.

### Example 3

| INGREDIENT | WT % | WT/g |
|---|---|---|
| ETHANOL 190C | 37.80 | 378.00 |
| 25% SELVOL 523 (PVA in water) | 10.00 | 100.00 |
| POLYSUGA NATE 100P (Poly(Decyl | 1.50 | 15.00 |
| Glucosides Hydroxypropyl Sulfonate) | | |
| GIOVEREZ P-0580 (Polyurethane-21 in | 40.00 | 400.00 |
| water)33% active | | |
| KAHL WAX 2442 P 5 (Carnauba wax) | 10.70 | 107.00 |
| TOTAL | 100.00 | 1000.00 |

### Procedure

1. ADD SELVOL TO GIOVAREZ WITH MIXING UNTIL UNIFORM

2. ADD ETOH, MIX

3. ADD WAX, MIX UNTIL UNIFORM

4. ADD SURFACTANT (POLYSUGARNATE), MIX UNTIL HOMOGENEOUS

5. QS WITH ETOH

### Example 4

Beach sand was poured into two separate 8×12 inch plastic troughs and heated in the oven to 60° C. Two subjects sprayed via pump spray the composition of Example 3 onto one hand and air dried to form a continuous film. The other hand was untreated. Both subjects placed each hand treated and untreated into the troughs of hot sand. The untreated hand became very hot and reached via laser thermometer 55° C.-50° C. while the treated hand remained at ambient temperature i.e. 21° C.-22° C.

### Example 5

| INGREDIENT | WT % | WT/g |
|---|---|---|
| BAYCUSAN C2000 (Polyurethane in | 88.00 | 484.00 |
| Alcohol) 40% | | |
| SPAN 80 HP-LQ-(MH) (Sorbitan Ester) | 1.00 | 5.50 |
| SPAN 20-LQ-(AP) (Sorbitan Ester) | 1.00 | 5.50 |
| KAHL WAX 2442 P 5 (Carnauba wax) | 10.00 | 55.00 |
| TOTAL | 100.00 | 550.00 |

### Procedure

1. ADD KAHL WAX TO BAYCUSAN, MIX

2. ADD SPAN80 AND SPAN20, MIX

3. QS WITH ETOH

### Laboratory Spec

### COLOR: YELLOW2

### ODOR: ALCOHOL

### THICK SMOOTH CREAMY LIQUID

### Example 6

The composition of Example 5 was tested in a similar manner according to Examples 2 and 4: results were similar regarding heat protection for treated glass slides and sand.

### Example 7

| INGREDIENT | WT % | WT/g |
|---|---|---|
| BAYCUSAN C2000 (Polyurethane in | 88.00 | 484.00 |
| Alcohol) | | |
| SPAN 80 HP-LQ-(MH) (Sorbitan Ester) | 1.00 | 5.50 |
| SPAN 20-LQ-(AP) (Sorbitan Ester) | 1.00 | 5.50 |
| KAHL WAX 2442 P 5 (Carnauba wax) | 10.00 | 55.00 |
| TOTAL | 100.00 | 550.00 |

### Procedure

1. ADD KAHL WAX TO BAYCUSAN, MIX

2. ADD SPAN80 AND SPAN20, MIX

3. QS WITH ETOH

### Laboratory Spec

### COLOR: YELLOW2

### ODOR: ALCOHOL

### THICK SMOOTH CREAMY LIQUID

### Example 8

The composition of Example 7 was tested as per examples 2 and 4: similar results were obtained.

### Example 9

Eighty parts of the composition from Example 7 were pressurized with twenty parts of propellant 152A to provide an aerosol spray with less than one minute drying time after application. Prior to pressurizing the concentrate was diluted 1:1 with ethanol.

### Example 10

The dried films from example 9 were again tested via the methods outlined in Examples 2 and 4 with similar results.

### Example 11

The aerosol product as per Example 9 was sprayed onto the feet of four subjects prior to walking on hot sand with estimated temperature of 130° F. The composition solidified in film form on the subjects' feet and the subjects did not express discomfort from heat for the entire six hour period on the beach with application/s repeated as needed.

### Example 12

The subjects used in Example 11 were exposed to swimming in sea water after application of the aerosol composition as per Example 11. An examination showed the films remained intact and water resistant.

### Example 13

Variations of the of the examples provided above have similar functionality include composition formulations of Polyurethane, Span 20, Span 80, Carnauba wax wherein Polyurethane ranged from about 70%-85% composition, Span 20 ranged from about 2%-3% composition, Span 80 ranged from about 2%-3% composition, and Carnauba wax ranged from about 10%-25% composition. In additional embodiments, the Polyurethane may comprise from 1 to 60 parts by weight of the total composition, the surfactants may be comprise 1 to 20 parts or 8 to 12 parts by weight of the total composition, the Carnauba wax may comprise 1 to 30 parts or 5 to 15 parts by weight of the total composition, and the solvent is 15 to 90 parts by weight of the total composition.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

1. A biocompatible thermal insulative composition comprising:
a. a film forming agent;
b. a phase transitioning material;
c. at least one surface active wetting agent or surfactant;
d. a solvent; and
wherein the thermal insulative composition has a first state in liquid, gel, or cream form, and a second state in solid film form.

2. The thermal insulative composition according to claim 1 wherein the film forming agent is polyvinyl acetate, the phase transitioning material is paraffin wax, the surfactant is sorbitan monooleate and the solvent is hydro alcoholic with alcohol/water ratios at 1-10 to 10-1.

3. The thermal insulative composition according to claim 1 wherein the said film forming agent is polyvinylalcohol, the phase transitioning material is beeswax, the surfactant is sodium lauryl sulfate and said solvent is isopropanol.

4. The thermal insulative composition according to claim 1 wherein the film forming agent polymer is polyurethane, the phase transitioning material is carnauba, the surfactant is sorbitan monooleate and the solvent is ethanol.

5. The thermal insulative composition according to claim 1 wherein the film forming agent comprises polyvinyl alcohol and polyurethane, the phase transitioning material comprises paraffin and carnauba, the surfactants are sorbitan monooleate and sorbitan monolaurate, and the solvent is ethanol.

6. The thermal insulative composition according to claim 1 wherein the film forming agent is polyurethane, the phase transitioning material is carnauba, the surfactants are sorbitan monooleate and sorbitan monolaurate and the solvent is ethanol.

7. The thermal insulative composition according to claim 6 wherein the film forming agent is present from 1 to 60 parts by weight of the total composition.

8. The thermal insulative composition according to claim 6 wherein the phase transitioning material is present at about 1 to 30 parts by weight of the total composition.

9. The thermal insulative composition according to claim 6 wherein the phase transitioning material is present at about 5 to 15 parts by weight of the total composition.

10. The thermal insulative composition according to claim 6 wherein the surfactants are present in a 1:1 ratio and comprise 1 to 20 parts by weight of the total composition.

11. The thermal insulative composition according to claim 6 wherein the surfactants are present in a 1:1 ratio and comprise 8 to 12 parts by weight of the total composition.

12. The thermal insulative composition according to claim 6 wherein the solvent is present at 15 to 90 parts by weight of the total composition.

13. The thermal insulative composition of claim 1 wherein the composition is in the second state applied to skin and the composition has a skin surface and an environment surface, wherein the composition comprises a thermal resistivity such that a temperature of the composition against the skin surface remains under 110° F. when the temperature against the environment surface is over 110° F.

14. The thermal insulative composition of claim 1 wherein the composition is in the second state applied to skin and the composition has a skin surface and an environment surface, wherein the composition comprises a thermal resistivity such that a temperature of the composition against the skin surface remains at ambient temperature when the temperature against the environment surface is above ambient temperature.

15. The thermal insulative composition of claim 1 comprising a delivery system configured to store and dispense the thermal insulative composition in liquid, gel, or cream form.

16. The thermal insulative composition of claim 16 wherein the delivery system comprises an aerosol can configured to dispense the thermal insulative composition in a pressurized spray.

17. A method of protecting skin from thermal damage comprising:
identifying a biocompatible thermal insulative composition wherein the thermal insulative composition has a first state in liquid, gel, or cream form, and a second state in solid film form and the thermal insulative composition comprising, a film forming agent, a phase transitioning material, at least one surface active wetting agent or surfactant, and a solvent;
applying the thermal insulative composition to the skin in the first state; and
allowing the thermal insulative composition to dry from the first state into the second state.

18. The method of claim 17 wherein the step of applying the thermal insulative composition further comprises the step of dispensing the thermal insulative composition from an aerosol can by spraying the thermal insulative composition in the first state onto the skin.

19. The method of claim 17 wherein the step of applying the thermal insulative composition further comprises the step of dispensing the thermal insulative composition from a roller ball container configured to roll a ball into a container to be exposed to and in contact with the thermal insulative composition in its first state, then roll the ball onto the skin further applying the thermal insulative composition from the ball onto the skin or the step of applying the thermal insulative composition further comprises the step of brushing the thermal insulative composition onto the skin.

20. The method of claim 17 further comprising the steps of reapplying the thermal insulative composition to the skin reinforce the thermal insulative composition in the second state or removing the thermal insulative composition in the second state from the skin.
